(19) United States
(12) Patent Application Publication
(10) Pub. No.: US 2025/0344779 A1
(43) Pub. Date: Nov. 13, 2025

(54) Underwear Garment with Integrated Absorbent Material Holder

(71) Applicants: Ebony Agee, Killeen, TX (US); Katara Lundy, Killeen, TX (US)

(72) Inventors: Ebony Agee, Killeen, TX (US); Katara Lundy, Killeen, TX (US)

(21) Appl. No.: 19/276,628

(22) Filed: Jul. 22, 2025

Related U.S. Application Data
(63) Continuation of application No. 17/885,613, filed on Aug. 11, 2022.
(60) Provisional application No. 63/280,656, filed on Nov. 18, 2021.

(51) Int. Cl. A41B 9/12; A61F 13/80

(52) U.S. Cl. CPC A41B 9/12; A61F 13/80

(57) ABSTRACT

A reusable female underwear garment having an integrated sanitary pad holder or any other absorbent material holder is disclosed. The underwear garment includes the holder positioned to cover the crotch area of a wearer such that a sanitary pad accommodated in the holder absorbs menstrual flow of the wearer. The holder is sewn and integrated to the underwear and includes tapered longitudinal elastic edges for accommodating the pad. The holder can have cuffs functioning as walls for providing security and stability to the pad. The holder keeps the pad in place and prohibits leakage and stains. The underwear garment can be a brief-style undergarment or any other style undergarment as per preferences of the manufacturers and users. The underwear garment is available in a variety of colors, styles, sizes, and designs.

### CROSS-REFERENCE TO RELATED APPLICATION

The present application is a continuation-in-part application of U.S. application Ser. No. 17/885,613 which was filed on Aug. 11, 2022, and claims priority to, and the benefit of, U.S. Provisional Application No. 63/280,656, which was filed on Nov. 19, 2021, both of which are incorporated herein by reference in their entirety.

### FIELD OF THE INVENTION

The present invention relates generally to clothing undergarments. More specifically, the present invention relates to a novel underwear garment for females or males that includes a disposable or sanitary pad holder sewn therein. More specifically, the underwear garment includes a holder that securely and removably accommodates and secures a disposable or sanitary pad thereby prohibiting the pad from shifting and wrinkling while being worn by a wearer. The pad remains in place and ensures contact or bodily touching with at least the perineum and crotch area and thus effectively absorbs menstrual flow or other fluid discharge without any leaks associated with use of disposable pads used in conjunction with conventional underwear. Accordingly, the present disclosure makes specific reference thereto. Nonetheless, it is to be appreciated that aspects of the present invention are also equally applicable to other like applications, devices, and methods of manufacture.

### BACKGROUND

By way of background, tampons are generally used by females for absorbing menstrual flow. Tampons are little cotton cylindrical pads designed to be inserted into the vagina with or without an applicator. Tampons come in different absorbencies to accommodate light to heavy periods. However, many females do not use tampons due to discomfort, excessive flow, and other preferences. Tampons can dry out the vagina and cause irritation and itching. Also, finding the right size of tampon takes some trial and error.

The biggest downside to wearing tampons is the risk of toxic shock syndrome (TSS). TSS is a rare but life-threatening complication caused due to certain types of bacterial infections and is primarily associated with use of tampons.

Often females use menstrual pads for absorbing menstrual flow. Generally, pads are rectangular absorbent material that adhere to the inside of underwear or are placed inside of the underwear garment. An advantage of the pads is that they are placed outside the vagina but they commonly get shifted out of place and wrinkle up towards the center. This shifting and wrinkling can result in embarrassing fluid leaks and permanent stains. Lack of security of positioning of the menstrual pads is a common problem and females desire a way of securely positioning their disposable menstrual pad.

Further, conventional undergarment/underwear causes a menstrual pad to directly touch the vagina of a wearer and thus the pad may cause irritation, infection, and more to the wearer. Individuals desire an improved way of wearing and using menstrual pads such that the pad does not directly touch the vagina of the wearer.

Conventional underwear for females especially panties, thongs, and others are ineffective in holding a menstrual pad. Further, all types of female underwear do not have a specific slot or holder positioned appropriately for accommodating a menstrual pad such that the pad is prohibited from shifting or losing its shape. Females desire an improved underwear for appropriately placing and positioning a menstrual pad inside the underwear for absorbing menstrual flow.

Therefore, there exists a long-felt need in the art for an improved undergarment for females and males that enables a disposable pad to be securely placed therein. There is also a long-felt need in the art for an underwear garment that prohibits shifting and wrinkling of the disposable pad placed therein. Additionally, there is a long-felt need in the art for an improved underwear garment that has a slot or holder for appropriately receiving and accommodating the disposable pad. Moreover, there is a long-felt need in the art for an underwear garment that maintains comfort of a wearer during a menstrual cycle or during the course of the day. Further, there is a long-felt need in the art for an improved underwear garment that prevents any leaks associated with using a disposable pad during the menstrual cycle or during incontinence. Finally, there is a long-felt need in the art for a user-friendly underwear garment that effectively holds the disposable pad and maintains its shape, size, and position inside the underwear garment.

The subject matter disclosed and claimed herein, in one embodiment thereof, comprises a brief-style female underwear garment. The underwear garment is configured to secure a sanitary pad in an integrated holder ensuring that the pad remains in place for absorbing menstrual flow and prohibiting any leaks associated with using a disposable pad during menstrual cycle. The female underwear garment further comprising a first leg opening and a second leg opening, a waistband, an absorbent material or sanitary pad holder sewn and positioned along a crotch covering region of the underwear, the holder has a pair of opposing longitudinal scams, a pair of lateral seams, an elastic cuff positioned along each of said longitudinal seams and lateral seams, wherein the cuffs create a shallow cavity for receiving and accommodating a menstrual pad, and the cuffs further function as walls for prohibiting movement and shrinkage of the menstrual pad.

In this manner, the menstrual pad-holding female underwear garment of the present invention accomplishes all of the forgoing objectives and provides females with a brief shaped undergarment that has a sewn holder for accommodating a menstrual pad, maintaining comfort, and prohibiting any leaks associated with using the menstrual pad. The holder is integrated to the underwear for receiving a pad and ensuring the pad remains in place thereby prohibiting leaks associated with using a disposable pad during a menstrual cycle

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a brief-style female underwear garment. The female underwear garment includes a first leg opening and a second leg opening, a waistband, an absorbent material holder positioned along a crotch region of the underwear, the holder has a pair of opposing longitudinal seams, a pair of lateral seams, an elastic cuff positioned along each of said longitudinal seams and lateral seams, wherein the cuffs create a cavity for receiving and accommodating a menstrual pad, and the cuffs further function as walls for prohibiting movement, shifting, and shrinkage of the menstrual pad.

In yet another embodiment, the absorbent material is a menstrual pad.

In yet another embodiment, the absorbent material is urine absorbing material.

In yet another embodiment, the absorbent material holder is positioned to cover at least a perineum area of a wearer.

In yet another embodiment, the absorbent material is removably placed in the holder.

In yet another embodiment, the cuffs are clastic.

In yet another embodiment, the absorbent material holder prevents shifting and shrinking of the absorbent material.

In yet another embodiment, a method for prohibiting leaks associated with using a disposable pad during a menstrual cycle is described. The method includes the step of providing a brief-style underwear garment for females, the underwear garment includes a holder sewn therein and positioned to cover at least a perineum area of a wearer; inserting a sanitary pad inside the holder; wearing the underwear; removing the sanitary pad from the holder after the pad has sufficiently absorbed menstrual flow; and, accommodating a new sanitary pad in the holder.

In yet another embodiment, a female underwear with a sewn menstrual pad holder is disclosed. The female underwear has the menstrual pad holder sewn inside the underwear such that the holder is positioned below the crotch area of the wearer, and the holder provides an enclosed space having an opening for inserting and removing a sanitary pad therein. The holder has clastic seams for accommodating pads of various sizes.

In yet another embodiment, a female underwear with a sewn menstrual pad holder is disclosed. The female underwear has the menstrual pad holder sewn inside the underwear such that the holder is positioned below the crotch area of the wearer, and the holder provides a securement area for placement and removal of a sanitary pad thereon. The holder can include clastic seams for accommodating pads of various sizes.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of the menstrual pad holder underwear of the present invention in accordance with the disclosed architecture;

FIG. 2 illustrates a rear perspective view of the brief-style underwear of FIG. 1 in accordance with the disclosed architecture;

FIG. 3 illustrates a planar top perspective view of the underwear of the present invention showing the position and shape of the holder inside the underwear in accordance with the disclosed architecture;

FIG. 4 illustrates a detailed view of the brief-style underwear of the present invention showing a sanitary pad being accommodated in the holder in accordance with the disclosed architecture;

FIG. 5 illustrates a perspective view of another embodiment of the sanitary pad holder that can be integrated inside the underwear of the present invention for accommodating a sanitary pad in accordance with the disclosed architecture; and

FIG. 6 illustrates a detailed view of the brief style underwear of the present invention showing a sanitary pad being accommodated in another holder in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved undergarment for females and males that enables a disposable pad to be securely placed therein. There is also a long-felt need in the art for an underwear garment that prohibits shifting and wrinkling of the disposable pad placed therein. Additionally, there is a long-felt need in the art for an improved underwear garment that has a slot or holder for appropriately receiving and accommodating the disposable pad. Moreover, there is a long-felt need in the art for an underwear garment that maintains comfort of a wearer during a menstrual cycle or during the course of the day. Further, there is a long-felt need in the art for an improved underwear garment that prevents any leaks associated with using a disposable pad during the menstrual cycle or during incontinence. Finally, there is a long-felt need in the art for a user-friendly underwear garment that effectively holds the disposable pad and maintains its shape, size, and position inside the underwear garment.

The present invention, in one exemplary embodiment, is a novel female underwear garment with a sewn or molded menstrual pad holder. The female underwear garment includes the menstrual pad holder sewn inside the underwear such that the holder bodily (i.e. directly) touches the crotch area of the wearer, and the holder provides an enclosed space having an opening for inserting and removing a sanitary pad therein. The holder has elastic seams for accommodating pads of various sizes.

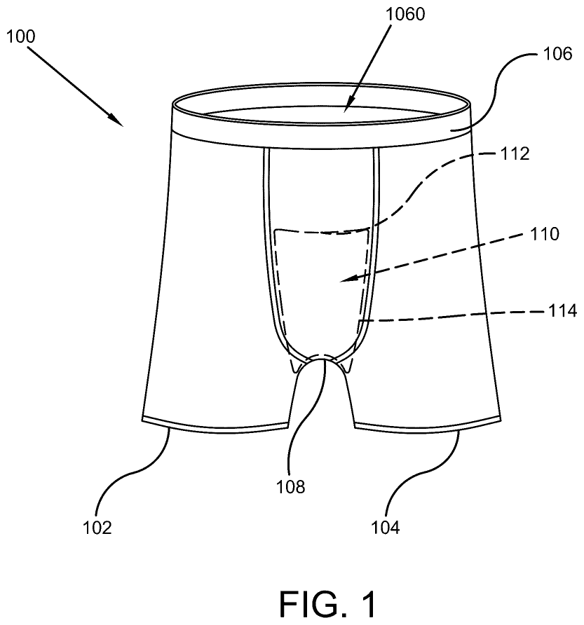

Referring initially to the drawings, FIG. 1 illustrates a front perspective view of one potential embodiment of the menstrual pad holder underwear of the present invention in accordance with the disclosed architecture. The menstrual pad holder underwear garment 100 of the present invention is preferably designed as a brief-style undergarment that has a holder sewn inside therein for holding a disposable menstrual pad. The underwear 100 is easy to wear and can be constructed of a breathable fabric such as but not limited to a perforated mesh material. Further, the underwear 100 enables female hygienic/menstrual pads to be placed securely inside the holder and thus prevents any leaks associated with using a pad during menstrual cycle by eliminating shifting and wrinkling of the pad.

More specifically, the underwear 100 has a right leg opening 102 and a left leg opening 104. The leg openings 102, 104 are used for receiving legs of a wearer enabling a user to wear the underwear 100. The underwear 100 further has a waistband 106 for securing around a waist of the wearer and creating an opening 1060 for wearing the underwear 100. As illustrated, along the crotch covering region 108 of the underwear 100, a menstrual pad holder 110 is positioned. The holder 110 is positioned inside the underwear 100 such that holder 110 is in bodily contact with the crotch area and more specifically, is in contact with at least a perineum area of the female wearer.

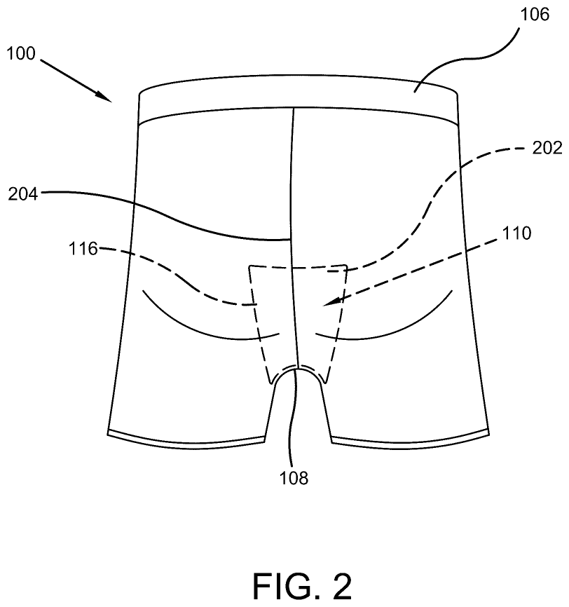

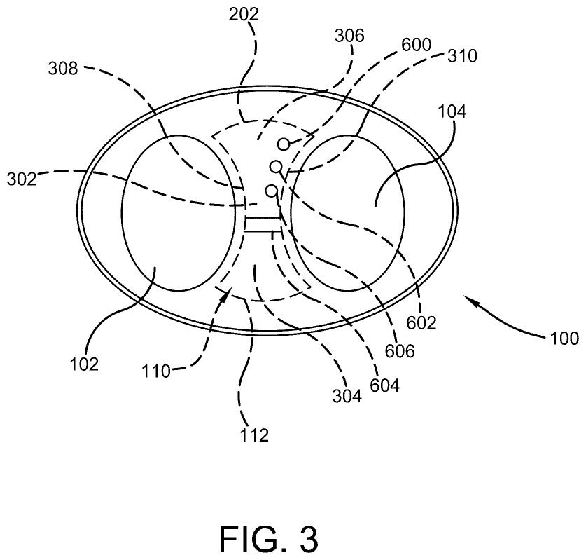

As illustrated in the front view, the holder 110 is dimensioned and designed to accommodate any conventional sanitary pad used for absorbing menstrual flow. The front end 112 of the holder 110 is positioned on front portion 114 inside the underwear and is symmetrically positioned to the back end 202 (shown in FIG. 2) of the holder 110. The portion of the holder 110 extending from the center of the crotch (shown in FIG. 3) covering region 108 to the front end 112 may also be referred to as front portion of the holder 110. In various embodiments, the holder 110 may be flat, tapered, and/or contoured with rise and fall to hug a user's anatomy.

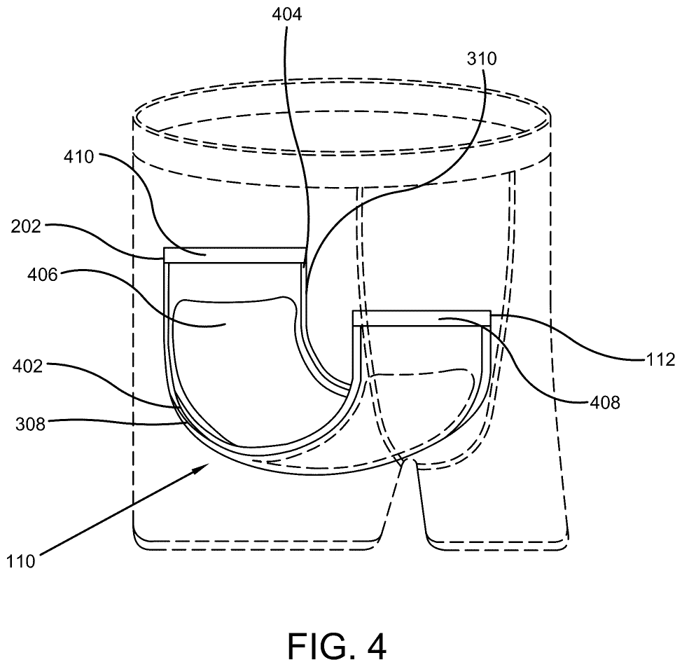

The holder 110 may be made of an elastic material and/or have elastic seams as illustrated in FIG. 4 that enables secure and stable placement of a sanitary pad even when a high movement activity is performed by the wearer of the underwear 100. Further, any type of absorbent articles for menstrual flow can be accommodated by the holder 110. The underwear 100 comes in a variety of sizes, designs, and materials making the underwear 100 suitable for everyday use.

The underwear 100 of the present embodiment has a unitary structure wherein the holder 110 is integrated inside the underwear 100. The holder 110 can be made of the same material as of the underwear 100 and alternatively can be made from a thin layer of an absorbent material. The absorbent material can be selected from the group consisting of wood pulp, cross-linked cellulosic fibers, synthetic fibers, and more.

FIG. 2 illustrates a rear perspective view of the brief style underwear 100 of the FIG. 1 in accordance with the disclosed architecture. As illustrated, the back portion 204 of the underwear 100 also has a portion of the holder 110 sewn inside. The back end 202 can be positioned symmetrical to the front end 112. The portion of the holder 110 extending from the center of the crotch covering region 108 to the back end 202 may also be referred to as rear portion 116 of the holder 110. In one embodiment, the holder 110 is not sewn and is fully integrated within the underwear 100 such that there are no external seams that produce outer grab on a user's anatomy. This sewless/seamless construction may be achieved via ultrasonic welding or other similar techniques.

It should be noted that the holder 110 includes a slightly tapered shape extending from the ends 112, 202 to the center of the crotch covering portion 108. The holder 110 has elastic seams that function as walls for holding a sanitary pad in the holder 110.

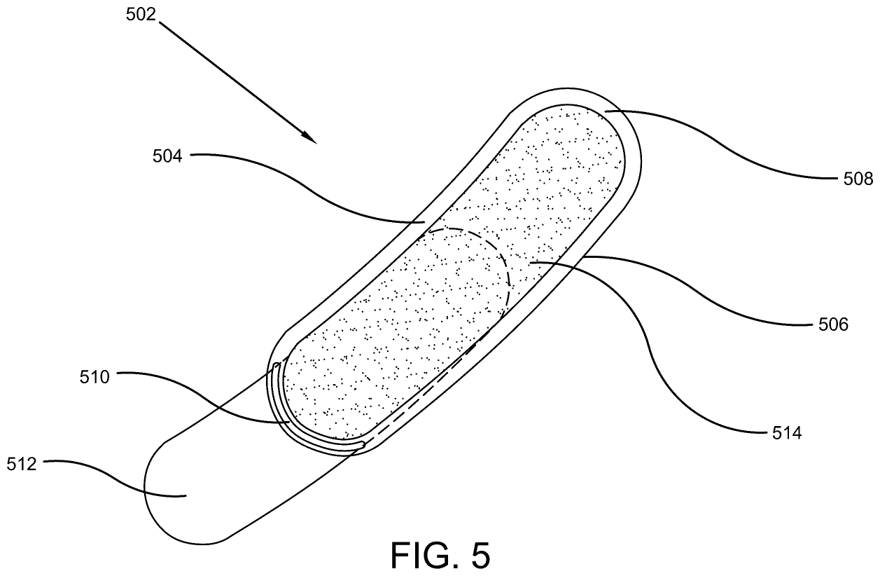

FIG. 3 illustrates a planar top perspective view of the underwear 100 of the present invention showing the position and shape of the holder 110 inside the underwear 100 in accordance with the disclosed architecture. As illustrated, the holder 110 is positioned between the leg openings 102, 104 such that holder 110 and the accommodated sanitary pad (shown in FIGS. 4 and 5) bodily touches or covers at least the perineum of a wearer and thus effectively absorbs the menstrual flow. The holder 110 prohibits shifting and shrinking of the accommodated sanitary pad even during brisk movements of the wearer, such as when the wearer is playing a sport. The front end 112 and the center 302 of the crotch covering region forms the front portion 304, and the rear end 202 and the center 302 of the crotch covering region forms the rear portion 306. The front end 112 and the rear end 202 are wider and the holder 110 tapers towards the center 302. The holder 110 is sewn and integrated inside the underwear 100 allowing a user to use the underwear 100 as a conventional underwear garment without holding a sanitary pad and can be used as menstrual flow absorbing device with a sanitary pad accommodated inside the holder 110.

The longitudinal tapered seams 308, 310 extending between the front end 112 and the rear end 202 are sewn and are clastic allowing sanitary pads of various sizes to be accommodated easily inside the holder 110.

FIG. 4 illustrates a detailed view of the brief style underwear 100 of the present invention showing a sanitary pad being accommodated in the holder 110 in accordance with the disclosed architecture. More specifically, FIG. 4 illustrates a detailed view of the holder 110. As illustrated, the longitudinal seams 308, 310 of the holder 110 have elastic cuffs 402, 404 extending slightly upward from the seams 308, 310 respectively. The cuffs 402, 404 function as walls and accommodate and secure a sanitary pad 406 placed in the holder 110. The front end 112 and the rear end 202 may also have similar cuffs 408, 410, respectively. The cuffs 402, 404, 408, 410 form a shallow cavity for accommodating the sanitary pad 406. The cuffs are elastic and sewn along the edges and provide stability to the sanitary pad 406 even in fast movement by a wearer of the female underwear 100. The sanitary pad 406 touches the perineum area of the wearer allowing the pad 406 to easily absorb menstrual flow of the wearer. Further, the pad 406 can be easily removed from the holder 110 and replaced with a new pad.

It should be noted that the cuffs can be bent and made flat when the underwear 100 is to be worn without accommodating the sanitary pad 406 inside the holder 110. The underwear 100 can be used as a conventional underwear garment for daily use and not just for menstrual flow cycles.

Further, the holder 110 is configured to accommodate any type of absorbent material and can be used by adults (i.e. females and males) for accommodating adult diaper material or other absorbent material to alleviate the problem of incontinence (i.e. urine or fluid discharge). This can obviate the need of using a complete diaper by an adult.

FIG. 5 illustrates a perspective view of another embodiment of the sanitary pad holder that can be integrated inside the female underwear 100 of the present invention for accommodating a sanitary pad in accordance with the disclosed architecture. In the present embodiment, the holder 502 has three sewn elastic edges including sewn elastic longitudinal edges 504, 506 and a rear end 508. An open end 510 is positioned on a front portion of the underwear 100 thereby allowing a user to insert a sanitary or disposable pad 512 inside the holder 502. The holder 502 may have a removable and absorbable cloth covering 514 for creating a cavity along with the sewn edges 504, 506, 508 for accommodating the disposable pad 512.

The sanitary or disposable pad 512 can be removed from the open end 510 as per preferences of the user. It should be noted that the length of the holder 502 is designed to cover crotch area (specifically at least the perineum area) of the wearer and thus protecting the wearer from embarrassing leaks, fluid or urine discharge, and permanent stains due to shifting and unstable positioning of the disposable pad 512.

The invention also discloses a method for securing a disposable or sanitary pad inside an underwear for prohibiting fluid leaks associated with using a disposable pad during menstrual cycle and/or bladder incontinence. The method includes the step of providing a brief-style underwear for females or males, the underwear having a holder sewn therein and positioned to cover at least the perineum area of a wearer; inserting a disposable or sanitary pad inside the holder; wearing the underwear; removing the disposable or sanitary pad from the holder after the pad has sufficiently absorbed menstrual flow or fluid discharge and accommodating a new disposable or sanitary pad in the holder.

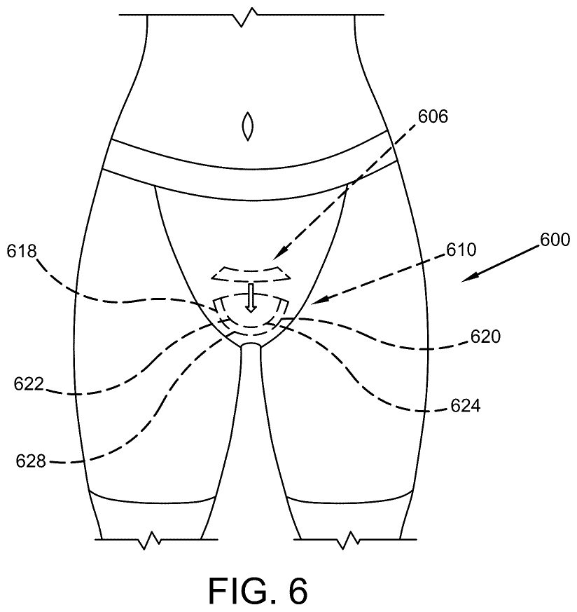

FIG. 6 illustrates a detailed view of the brief style underwear 600 of the present invention showing a sanitary pad being accommodated in the holder 610 in accordance with the disclosed architecture. More specifically, FIG. 6 illustrates a view of the holder 610. As illustrated, the longitudinal seams 618, 620 of the holder 610 can include cuffs 622, 624 extending slightly upward from the seams 618, 620 respectively. The cuffs function as walls and accommodate a sanitary pad 606 placed on top of the holder 610. The front end and the rear end can also have a front cuff 628 and a rear cuff (not illustrated), respectively. The cuffs 622, 624, 628 form a border for accommodating the sanitary pad 606 therebetween. The cuffs 622, 624, 628 are clastic and sewn along the edges and provide stability to the sanitary pad 606 even during strenuous movements of a wearer of the female underwear 600. The sanitary pad 606 touches the perineum area of the wearer allowing the pad 606 to easily absorb menstrual flow of the wearer. Further, the pad 606 can be easily removed from the holder 610 and replaced with a new pad.

It should be noted that the cuffs 622, 624, 628 can be bent and made flat when the underwear 600 is to be worn without accommodating the sanitary pad 606 inside the holder 610. The underwear 600 can be used as a conventional underwear garment for daily use and not just for menstrual flow cycles.

Further, the holder 610 is configured to accommodate any type of absorbent material and can be used by adults (i.e. females and males) for accommodating adult diaper material or other absorbent material to alleviate the problem of incontinence (i.e. urine or fluid discharge). This can obviate the need of using a complete diaper by an adult.

The sanitary pad holder underwear garment 100, 600 of various embodiments of the present invention includes a variety of colors, sizes, and designs and can be worn daily. Any of conventional manufacturing and sewn techniques like zig zag technique can be implemented to manufacture the underwear garment 100, 600. The underwear garment 100, 600 prohibits any leaks associated with using a disposable pad during a menstrual cycle or urine incontinence. Insertion and removal of a sanitary pad is extremely easy and the sanitary pad is prohibited from shifting or wrinkling.

In one embodiment, any portion of the underwear 100 may be comprised of a moisture barrier 600. In one embodiment, the barrier 600 is integrated between (but not limited to) the pad 406 and the rest of the underwear 100 to prevent bodily fluid from soaking through the undergarment 100. The barrier 600 may be comprised of materials such as but not limited to polyurethane laminate (PUL), thermoplastic polyurethane (TPU), or other medical-grade waterproof fabrics that may be laminated or ultrasonically bonded to the underside of the holder region to ensure complete coverage of the perineum and lower crotch area of the user.

In a further embodiment, any area of the undergarment 600 may be comprised of an odor-neutralizing layer 602 comprised of materials such as but not limited to activated carbon fabric, bamboo charcoal-infused textiles, or zeolite-treated fibers. In one embodiment, the layer 602 may be positioned between (but not limited to) the inner surface of the undergarment 100 and the sanitary pad 406 to continuously absorb volatile compounds released during menstrual or incontinence use.

In an additional embodiment, the undergarment 100 may be comprised of at least one wicking structure 604 such as but not limited to micro-channeled or ribbed wicking structures designed to promote uniform fluid distribution across the surface of the sanitary pad 406, wherein the structures may be molded, stitched, or embossed and located anywhere on the undergarment 100 and/or on any component. The structures 604 may be formed in various patterns such as but not limited to herringbone, diamond, or longitudinal grooves based on anatomical fluid flow patterns and wearer movement.

Additionally, the undergarment 100 may be comprised of an anti-microbial coating 606 to improve hygiene and reduce the risk of odor, irritation, or infection. The coating 606 may be applied to any portion of the undergarment and may be comprised of but is not limited to a silver ion infusion, copper oxide impregnation, or antibacterial polymer coatings (e.g., chitosan or triclosan derivatives) to inhibit the growth of bacteria, yeast, and fungi associated with menstrual blood and bodily fluids.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “menstrual pad holder underwear”, “absorbent material holder underwear”, “underwear”, “underwear garment”, “sanitary pad holder underwear”, “brief style underwear” and “female underwear” are interchangeable and refer to the female underwear 100 with integrated absorbent material holder 110 of the present invention.

Notwithstanding the forgoing, the female (or male) underwear garment 100, 600 with integrated absorbent material holder 110, 610 of the present invention can be of any suitable size and configuration as is known in the art without affecting the overall concept of the invention, provided that it accomplishes the above-stated objectives. One of ordinary skill in the art will appreciate that the female/male underwear garment 100, 600 with integrated absorbent material holder 110, 610 as shown in the FIGS. are for illustrative purposes only, and that many other sizes and shapes of the female/male underwear 100, 600 with integrated absorbent material holder 110, 610 are well within the scope of the present disclosure. Although the dimensions of the female/male underwear garment with integrated absorbent material holder 110, 610 are important design parameters for user convenience, the female/male underwear garment 100, 600 with integrated absorbent material holder 110, 610 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An underwear garment comprising:
a holder sewn to an inside of said underwear garment for holding a menstrual pad, wherein said holder secures the menstrual pad for prohibition of shifting and wrinkling of the menstrual pad;
a breathable fabric;
a wicking structure;
a moisture barrier layer;
an odor control layer;
a right leg opening and a left leg opening for receiving a pair of legs of a wearer; and
a waistband for securing around a waist of the wearer, wherein said holder covers a crotch region of the wearer and is in bodily contact with said crotch region, and further wherein said crotch region includes at least a perineum area.

2. The underwear garment of claim 1, wherein the odor control layer is comprised of an activated carbon fabric, a bamboo charcoal-infused textile, or a zeolite-treated fiber.

3. The underwear garment of claim 2, wherein said holder comprises a front portion symmetrical to a rear portion, and further wherein said front portion and said rear portion are centered in said crotch region.

4. The underwear garment of claim 1, wherein said holder comprises a first longitudinal elastic seam and a second opposing longitudinal elastic seam for securing the menstrual pad therebetween.

5. The underwear garment of claim 4, wherein said holder comprises a rear elastic seam for positioning the menstrual pad.

6. The underwear garment of claim 5, wherein said underwear garment and said holder comprise a material selected from the group consisting of a wood pulp, a plurality of cross-linked cellulosic fibers, and a plurality of synthetic fibers.

7. The underwear garment of claim 6, wherein said underwear garment comprises a first layer thickness and said holder comprises a second layer thickness, and further wherein said second layer thickness is less than said first layer thickness.

8. The underwear garment of claim 5, wherein said holder comprises a tapered shape tapering from opposing ends of said holder toward a center area of said holder.

9. The underwear garment of claim 8, wherein said first longitudinal elastic seam comprises a first elastic cuff there along and said second opposing longitudinal elastic seam comprises a second elastic cuff there along, and further wherein said first elastic cuff and said second elastic cuff for securing the menstrual pad therebetween.

10. The underwear garment of claim 9, wherein said rear elastic seam comprises a rear elastic cuff there along for securing the menstrual pad therebetween.

11. The underwear garment of claim 10, wherein said holder comprises a front elastic seam having a front elastic cuff there along for securing the menstrual pad therebetween.

12. An underwear garment comprising:
a moisture barrier;
an odor control layer;
an antimicrobial coating;
a disposable pad comprised of an absorbent material for absorbing a fluid discharge from a wearer;
a holder sewn to the inside of said underwear garment for holding the disposable pad, wherein said holder secures said disposable pad for prohibition of shifting and wrinkling of said disposable pad when worn by the wearer;
a right leg opening and a left leg opening for receiving a pair of legs of the wearer; and
a waistband for securing the underwear garment around a waist of the wearer, wherein said holder covers a crotch region of the wearer and is in bodily contact with said crotch region.

13. The underwear garment of claim 12, wherein the antimicrobial coating is comprised of a silver ion infusion, a copper oxide impregnation, or an antibacterial polymer coating.

14. The underwear garment of claim 13, wherein said holder comprises a front portion symmetrical to a rear portion, and further wherein said front portion and said rear portion are centered in said crotch region.

15. The underwear garment of claim 14, wherein said holder comprises a first longitudinal elastic seam and a second opposing longitudinal elastic seam for securing said disposable pad therebetween.

16. The underwear garment of claim 15, wherein said holder comprises a rear elastic seam for positioning said disposable pad.

17. The underwear garment of claim 15, wherein said first longitudinal elastic seam comprises a first elastic cuff there along and said second opposing longitudinal elastic seam comprises a second elastic cuff there along, and further wherein said first elastic cuff and said second elastic cuff for securing said disposable pad therebetween.

18. An underwear garment comprising:
a holder sewn to an inside of said underwear garment for holding a disposable pad, wherein said holder secures said disposable pad for prohibition of shifting and wrinkling of said disposable pad when worn by a wearer;
a right leg opening;
a left leg opening;
a moisture barrier;
an odor control layer;
an antimicrobial coating;
a wicking structure;
a waistband for securing the underwear garment around a waist of the wearer;
wherein said holder covers a crotch region of the wearer and is in bodily contact with said crotch region;
wherein said disposable pad comprises an absorbent material for absorbing fluid discharge from the wearer; and
further wherein said holder includes a first longitudinal elastic seam, a second opposing longitudinal elastic seam, and a rear elastic seam for securing said disposable pad therebetween.

19. The underwear garment of claim 18, wherein said first longitudinal elastic seam comprises a first elastic cuff there along and said second opposing longitudinal elastic seam comprises a second elastic cuff there along, and further wherein said first elastic cuff and said second elastic cuff for securing said disposable pad therebetween.

20. The underwear garment of claim 19, wherein said rear elastic seam comprises a rear elastic cuff there along for securing said disposable pad therebetween.
